(19) United States
(12) Patent Application Publication
(10) Pub. No.: US 2007/0288077 A1
(43) Pub. Date: Dec. 13, 2007

(54) SELF-ANCHORING ELECTRICAL LEAD WITH MULTIPLE ELECTRODES

(76) Inventors: Cherik Bulkes, Sussex, WI (US); Stephen Denker, Mequon, WI (US)

Correspondence Address: QUARLES & BRADY LLP, 411 E. WISCONSIN AVENUE, SUITE 2040, MILWAUKEE, WI, 53202-4497, US

(21) Appl. No.: 11/759,513

(22) Filed: Jun. 7, 2007

Related U.S. Application Data
(60) Provisional application No. 60/811,539, filed on Jun. 7, 2006.

(51) Int. Cl. A61N 1/05

(52) U.S. Cl. 607/116

(57) ABSTRACT

An apparatus provides an electrical interface with a lumen in a body of an animal. The apparatus has a self-anchoring lead structure for implantation inside the lumen and includes at least two insulated conductors each connected to a separate electrode. Each electrode has an associated shape memory material and a rounded terminus to grip the lumen wall for anchoring the lead when properly positioned. The conductor for each electrode also is connected to a control circuit that programmably selects electrodes for electrically interfacing with the lumen. The self-anchoring lead structure has a contracted state for insertion into the animal and an expanded stated in which the electrode termini engage a wall of the lumen.

### CROSS-REFERENCE TO RELATED APPLICATIONS

This application claims benefit of U.S. Provisional Patent Application No. 60/811,539 filed on Jun. 07, 2006.

### STATEMENT REGARDING FEDERALLY SPONSORED RESEARCH OR DEVELOPMENT

Not Applicable

### BACKGROUND OF THE INVENTION

1. Field of invention

The present invention relates to implantable devices, which deliver energy to stimulate tissue to provide therapy to and/or sense electrical signals from the tissue of an animal, and more particularly to a novel self-anchoring lead that provides an electrical interface at multiple contacts with the tissue of an animal.

2. Description of the Related Art

A common remedy for a patient with a physiological ailment is to implant an electrical stimulation device. An electrical stimulation device is a small electronic apparatus that stimulates an organ or part of an organ. It includes a pulse generator, implanted in the patient, which produces electrical pulses to stimulate the organ. Electrical leads extend from the pulse generator to electrodes placed adjacent to specific regions of the organ, which when electrically stimulated provide therapy to the patient.

An improved apparatus for physiological stimulation of a tissue includes a wireless radio frequency (RF) receiver implanted as part of a transvascular platform that comprises at least one electrode that is connected to the wireless RF receiver and an electronic capsule containing a stimulation circuitry. The stimulation circuitry receives the radio frequency signal and from the energy of that signal derives an electrical voltage. The electrical voltage is applied in the form of suitable waveforms to electrodes, thereby stimulating the tissue.

As mentioned above, a lead with one or more electrodes forms an integral part of the stimulation system. A lead is an insulated wire that is connected to an implanted device. Leads need to be extremely flexible in order to withstand the twisting and bending caused by body movement and movement by the organ itself. A lead is usually designed to perform at least one of stimulating the organ with an electrical waveform and sensing electrical activity of an organ back to the device.

A lead usually includes a connector, a lead body and a securing mechanism. The connector is the portion of the lead that is inserted into the connector block on the device. The body of the lead has an insulated metal wire that carries electrical energy from the device to the organ in the stimulation mode or from the organ to the device in the sensing mode. The securing mechanism is near the tip of the lead and holds the lead to the organ. At least one electrode is located at the tip of the lead. The electrode delivers the electrical energy from the device to the organ tissue. The electrode may also detect the organ's electrical activity. One or more leads are typically used, depending on the medical condition treated and the patient's response to the treatment.

A lead is placed inside or outside the organ or tissue to be stimulated. For most adults, a lead is usually inserted through a vein and guided close to or into the organ. This is called a transvenous lead because it is inserted through a vein.

Sometimes the lead is attached to the outside the organ, especially for children with growing bodies. This lead is also used when another surgery is being done and the exterior of the organ is easy to reach.

Regardless of whether a lead is placed on the inside or outside the organ, the location where the lead touches the organ naturally produces an inflammatory response. This response is similar to what is observed when skin is scraped: the area around the scrape gets inflamed and may result in a scar as body repairs itself. When a lead is placed in an organ, a similar response occurs. By placing a medication, called a steroid, at the tip of the lead, this inflammation can be reduced. When the lead is placed in or on the organ, the medication is released and the build-up of scar tissue between the electrode and the organ tissue is minimized. Reducing the amount of scar tissue helps the stimulation system work more efficiently.

An approach to the implantation of an intravenous lead is the use of a flexible guide wire along which the lead is slid to its destination. The guide wire, entrained within a lumen of the lead body, is advanced along a transvenous lead feed path to the desired position within the target vein. The lead is then pushed or advanced along the guide wire until the distal tip thereof reaches the desired position. The guide wire is then retracted and removed from the lead body.

Many presently available intravascular leads are multi-polar in which—besides an electrode at the tip—one or more ring electrodes are incorporated in the distal end portion of the lead for transmitting electrical stimulation pulses from the pulse generator to the organ and/or to transmit naturally occurring sensed electrical signals from the organ to the pulse generator. Thus, by way of example, in a typical bipolar lead having a tip electrode and a ring electrode, two concentric conductor coils with insulation in between are carried within the electrically insulating sheath. One of the conductor coils connects the pulse generator with the tip electrode while the other conductor coil, somewhat shorter than the first conductor coil, connects the pulse generator with the ring electrode positioned proximally of the tip electrode. To reduce the outside diameter of multi-polar leads, the individual conductor wires are each insulated and instead of being coaxial or concentric, all of the conductor wires are wound on the same diameter into a coil. In a multi-polar lead employing this technique, the various wires are interleaved in a single solenoidal coil, along the same coil diameter, thereby helping to reduce the overall diameter of the lead.

To further reduce the outside diameter, lead bodies having multiple lumens have been developed. In place of coils wound from wire, multi-strand, braided cable conductors may be used to connect the pulse generator at the proximal end of the lead with the tip and ring electrodes at the distal end of the lead. In some existing lead assemblies, a combination of a coil conductor and one or more cable conductors are utilized. In this case, the coil conductor is typically passed through a non-coaxial lumen, which is a lumen that is offset from the longitudinal axis of the lead body. Multi-lumen lead bodies may also carry defibrillation electrodes and associated combinations of coil or cable conductors as part of the stimulation apparatus.

Despite the advances made in the art, there remains a need for improved body implantable, stimulation/sensing leads and related lead systems that are especially suited for transluminal stimulation/sensing systems. This is specifically to ensure that the electrodes make lumen wall contact with minimal adverse impact on that wall.

### SUMMARY OF THE INVENTION

One objective of the invention is to provide a self-anchoring lead for providing an electrical interface within a lumen in the body of an animal. The lead contains a lead structure to be implanted inside the lumen with at least two insulated conductors, each of which is connected to an electrode to electrically interface with a tissue near the lumen wherein the electrode has an associated shape memory material and the electrode has a rounded terminus to grip the body lumen wall for anchoring the lead when released. The conductor from each of the plurality of electrodes is also connected to a control circuit wherein the control circuit programmably selects electrodes for electrically interfacing with the lumen.

More specifically, a self-anchoring lead provides an electrical interface with a blood vessel of an animal. The lead includes a lead body to be implanted inside the blood vessel with a plurality of coiled insulated conductors. In a preferred embodiment, the insulated conductors are coiled about a common axis, however they may be coiled individually along different axes. Each insulated conductor is connected to an electrode to electrically interface with tissue near the blood vessel. The electrode has an associated shape memory material. The electrode has a rounded terminus to grip the blood vessel wall for anchoring the lead when released by pulling a sheath holding the electrode in a collapsed state. The lead structure has an internal lumen for placing a guidewire or other placement implement. Optionally, an external, biocompatible layer may cover the lead structure. The conductor from each of the plurality of electrodes is also connected to a control circuit, wherein the control circuit programmably selects electrodes for electrically interfacing with the blood vessel.

A method of providing an electrical interface with a lumen in a body of an animal includes implanting a self-anchoring lead in the lumen by inserting the lead in a collapsed state through an opening in the lumen and advancing the lead adjacent to a desired interface site. The self-anchoring lead comprises an expandable portion with a plurality of electrodes that electrically contact the lumen wall. Each electrode has an associated shape memory material and a rounded terminus. The self-anchoring electric lead also has a non-expandable portion that includes a plurality of coiled, insulated conductors connected to the electrodes. Once properly located, the expandable portion of the lead is released by pulling a sheath that confined that portion in a collapsed state. Upon being deployed in this manner, the latter portion of the lead expands so that the rounded termini grip the lumen wall thereby anchoring the lead.

### BRIEF DESCRIPTION OF DRAWINGS

FIG. 1 schematically depicts external and internal subsystems of a wireless transvascular platform for animal tissue stimulation;

FIG. 2 is a block schematic circuit diagram of the internal subsystem;

FIGS. 3A and 3B respectively show side and end views of a first type of prior art ring electrode and lead configuration;

FIGS. 4A and 4B respectively depict side and end views of a second type of prior art ring electrode and lead configuration;

FIG. 5 is shows a self-anchoring lead according to the present invention deployed in a lumen in the body of an animal;

FIG. 6 shows different configurations of the terminus of the electrodes of the self-anchoring lead;

FIG. 7 illustrates internal details of the electrode portion of the lead in the case of an insulated conductor with shape memory;

FIG. 8 shows internal details of the electrode portion of the lead in the case of an insulated conductor with an associated shape memory wire; and

FIG. 9 depicts internal oblique section of an expandable part of the lead;

FIG. 10 is an external cross section of the lead at an expandable part; and

FIG. 11 is shows a self-anchoring lead is a contracted state during insertion into the lumen in the body of an animal.

### DETAILED DESCRIPTION OF THE INVENTION

Although the present invention is being initially described in the context an intravascular radio frequency energy powered cardiac stimulator, the novel self anchoring lead can be used in a conventional cardiac rhythm management device for stimulation and/or sensing. In addition to cardiac applications, the self anchoring lead can provide brain stimulation for treatment of obsessive/compulsive disorder or Parkinson's disease, for example. The electrical stimulation and/or sensing using the present lead also may be applied to muscles, the spine, the gastro/intestinal tract, the pancreas, and the sacral nerve. The lead may also be used for GERD treatment, endotracheal stimulation, pelvic floor stimulation, treatment of obstructive airway disorder and apnea, molecular therapy delivery stimulation, chronic constipation treatment, and electrical stimulation for bone healing.

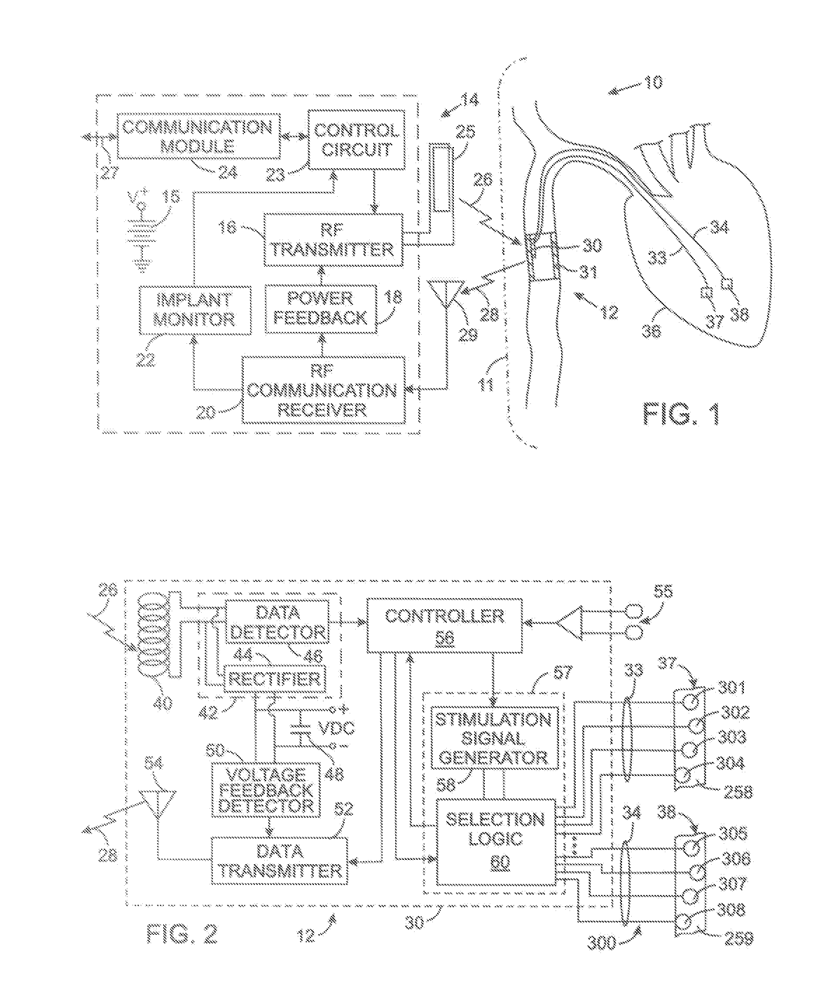

With initial reference to FIG. 1, a transvascular platform 10 for tissue stimulation includes an extracorporeal power source 14 and a stimulator 12 implanted inside the body 11 of an animal. The extracorporeal power source 14 communicates with the implanted stimulator 12 via wireless signals. The extracorporeal power source 14 includes a rechargeable battery 15 that powers a transmitter 16 which sends a first radio frequency (RF) signal 26 via a first transmit antenna 25 to the stimulator 12. The first RF signal 26 provides electrical power to the stimulator 12. The transmitter 16 pulse width modulates the first RF signal 26 to control the amount of power being supplied. The first radio frequency signal 26 also carries control commands and data to configure the operation of the stimulator 12.

The implanted stimulator 12 has an electronic circuit 30 that is mounted on a circuit carrier 31 and includes an radio frequency transceiver and a tissue stimulation circuit similar to that used in previous pacemakers and defibrillators. That circuit carrier 31 is positioned in a large blood vessel 32, such as the inferior vena cava (IVC), for example. One or more, electrically insulated electrical cables 33 and 34 extend from the electronic circuit 30 through the coronary blood vessels to locations in the heart 36 where pacing and sensing are desired. The electrical cables 33 and 34 terminate at stimulation electrodes located on electrode assemblies 37 and 38 at those locations. Each electrode assembly 37 and 38 has a plurality of contact electrodes, as will be described.

With reference to FIG. 2, the electronic circuit 30 of the implanted stimulator 12 has a first receive antenna 40 tuned to pick-up a first RF signal 26 from the extracorporeal power source 14. The signal from the first receive antenna 40 is applied to a discriminator 42 that separates the received signal into power and data components. Specifically, a rectifier 44 functions as a power circuit which extracts energy from the first RF signal to produce a DC voltage (VDC) that is applied across a storage capacitor 48 from which electrical power is supplied to the other components of the stimulator 12. The DC voltage is monitored by a voltage feedback detector 50 that provides an indication of the capacitor voltage level to a data transmitter 52 which sends that indication from a second transmit antenna 54 via the second radio frequency signal 28 to the extracorporeal power source 14.

Commands and control data carried by the first RF signal 26 are extracted by a data detector 46 in the stimulator 12 and fed to an analog, digital or hybrid controller 56. That controller 56 receives physiological signals from sensors 55 implanted in the animal. In response to the sensor signals, the controller 56 activates a stimulation circuit 57 that comprises a stimulation signal generator 58 which applies a stimulation voltage via selection logic 60 to the electrode assemblies 37 and 38, thereby stimulating the adjacent tissue in the animal.

Referring again to FIG. 1, the extracorporeal power source 14 receives the second radio frequency signal 28 carrying data sent by the stimulator 12. That data include the supply voltage level as well as physiological conditions of the animal, status of the stimulator and trending logs, that have been collected by the implanted electronic circuit 30, for example. To receive that second RF signal 28, the extracorporeal power source 14 has a radio frequency communication receiver 20 connected to a second receive antenna 29. A power feedback module 18 extracts data regarding the supply voltage level in the stimulator 12 to control the generation of the first RF signal 26 accordingly. An implant monitor 22 extracts stimulator operational data from the second RF signal 28, which data are sent to a control circuit 23. An optional communication module 24 may be provided to exchange data and commands via a communication link 27 with other external apparatus (not shown), such as a programming computer or patient monitor so that medical personnel can review the data or be alerted when a particular condition exists. The communication link 27 may be a wireless link such as a radio frequency signal or a cellular telephone connection.

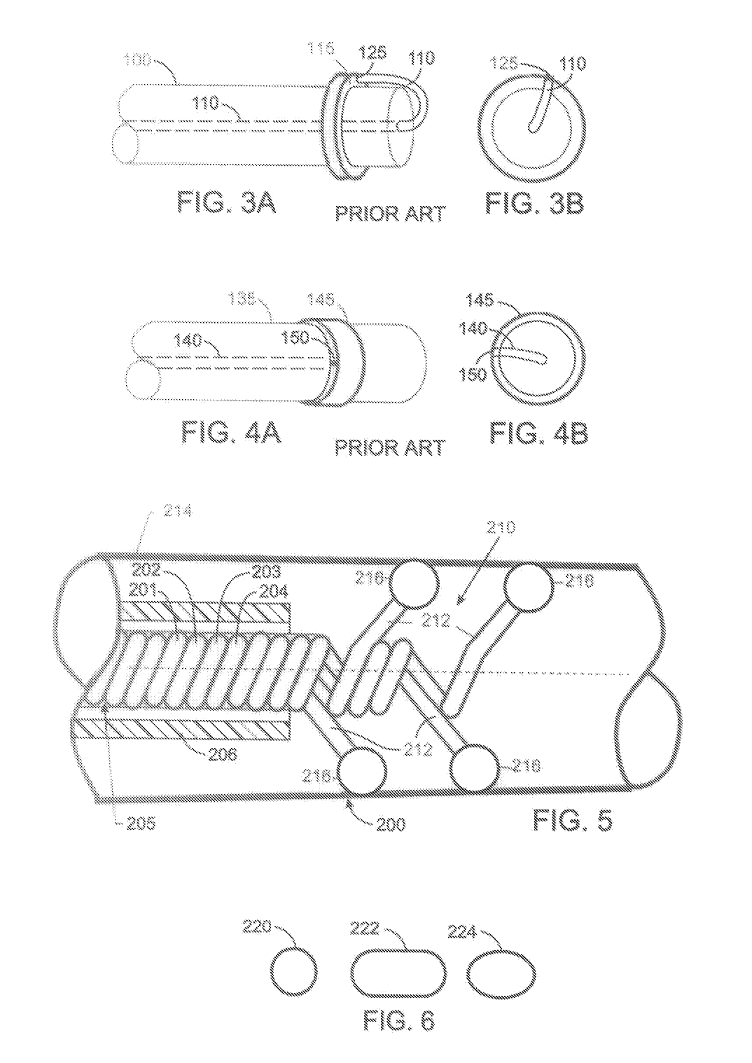

FIG. 3 shows a prior art stimulation lead configuration. The lead body 100 has an insulated conductor 110 connected to a signal generator (not shown) and terminating on the ring electrode 115 after looping out of the end of the lead. The conductor 110 is welded at contact 125 to the ring electrode. While the contact is secure in this configuration, it may result in vessel wall damage.

FIG. 4 illustrates an alternative configuration of the prior art. The lead body 135 has an insulated conductor 140 connected to a signal generator (not shown) and terminating on the ring electrode 145 directly without looping out of the lead. The conductor 140 is welded at contact 150 to the ring electrode. While the contact 150 is secure in this configuration, it may also result in damage to the wall of the lumen in which it is implanted.

FIG. 5 depicts a novel self-anchoring lead 200 that has a non-expandable portion 205 which includes a plurality of insulated conductors 201-204 that are spirally wound side by side in an interleaved manner to form a cylindrical coil. Four insulated conductors 201, 202, 203 and 204 are shown in a coiled cylindrical formation in this exemplary lead 200. The conductors 201-204 terminate at electrodes 212 in an expandable portion 210 of the lead 200. The electrodes 212 contact the lumen wall 214 when the lead is deployed in an animal and anchor the lead against being displaced under usual conditions. At the same time it is important to prevent any local injury or irritation to the tissue due to friction. The injury or irritation in the present invention is minimized by the electrode termini 216 that are in contact with the lumen wall having a rounded shape with a diameter that is larger than the diameter of the conductor associated. A five times larger diameter is preferred.

The self-anchoring lead 200 has an outer sheath 206 that for implantation of the lead extends over the expandable portion 210 and confines the electrodes 212 in a collapsed state within the sheath as seen in FIG. 11. After the lead 200 has been fed through the lumen so that the expandable portion 210 is located adjacent the site to be stimulated, the sheath 206 is pulled back to slide away from the tip of the lead, thereby exposing the electrodes 212 as seen in FIG. 5. This enables the electrodes 212 to expand radially outward as illustrated, that their termini 216 engage the lumen wall 214. After the lead 200 is secured in place, the sheath 206 may be removed from the animal.

In FIG. 6, three alternatives for the rounded shape of the termini 216 of the electrodes 212 are shown. These exemplary alternatives are spherical 220, capsule-like 222 or ellipsoidal 224, however other shapes also can be employed.

Since the electrodes are designed for deployment at a desired site in a lumen, they need to have a smaller size which enables the lead to be inserted into that site. This need necessitates the use of shape memory materials associated with the electrodes. The shape memory material may be part of the conductor or an external element that is attached to the insulated conductor by shrink-wrapping the polymer layer around the conductor-electrode combination. Accordingly, each of these embodiments is described further with illustrative examples.

With reference to FIG. 7, a first embodiment comprises an electrode 236 with an internal conductor 230 formed by a conductive material with shape memory, for example, stainless steel or a nickel-cobalt based alloy such as MP35N (trademark of SPS Technologies, Inc.). The shape memory conductor 230 is covered with an insulation layer 232 and is directly in connected to the electrode terminus 234. The insulated conductor 230 may be surrounded by a layer 235 of biocompatible material forming the external surface of the electrode 236. A biocompatible material is a substance that is capable of being used in the human body without eliciting a rejection response from the surrounding body tissues, such as inflammation, infection, or an adverse immunological response.

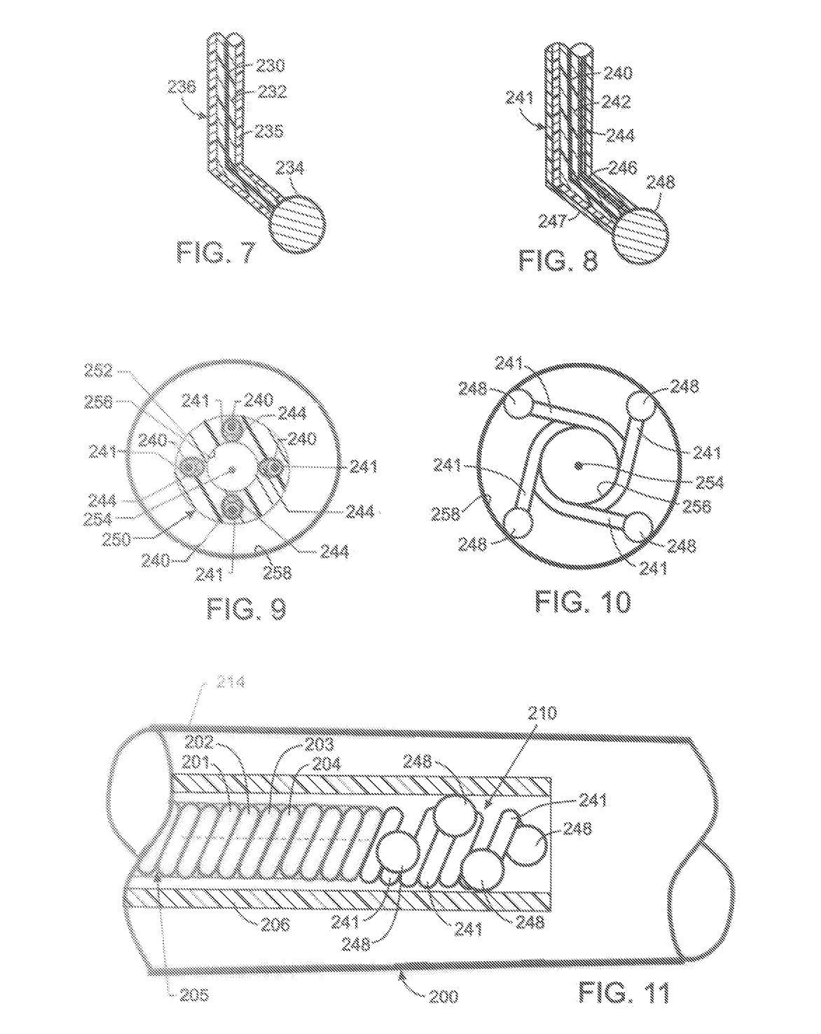

In a second embodiment of an electrode 241 shown in FIG. 8, the conductor 240 is a high conductivity material, for example, a conductive alloy such as MP35N®, stainless steel, a plated conductor such as a silver plated conducting wire, that is connected to a rounded electrode terminus 248. The conductor 240 is covered by an insulation layer 242 with a shape memory wire 244 placed next to the insulated conductor. The shape memory wire 244 may be a metal alloy such as for example Nitinol, stainless steel, MP35N® to mention only a few thus being electrically conductive, or it may be made of a non-conductive shape memory material, such as certain well-known polymers and ceramics. The shape memory material 244 and the insulation layer 242 are shrink-wrapped using a suitable polymer material 246, for example, polyurethane, such that the shrink-wrapped combination now has shape memory properties. The electrode 241 has an outer biocompatible layer 247. The second embodiment of the electrode 241 is incorporated into a lead 250, as illustrated in FIG. 9 which depicts an oblique cross section there through. This lead 250 contains four of the electrodes 241 that have insulated conductors 240 and adjacent shape memory wires 244. As described previously, the combination of an insulated conductor and the shape memory wire is shrink-wrapped by a suitable polymer. An optional outer biocompatible layer 252 may be used if the shrink-wrap material itself is not biocompatible. The internal lumen 256 of the lead 250 typically is provided to receive a guidewire 254 or other work implement. Because the electrode termini 248 are not visible in this oblique sectional view, the lead 250 appears to be floating in the body lumen 258.

The anchoring mechanism is shown in FIG. 10 where four expanded electrodes 241 have electrode termini 248 in contact with the lumen 258 in the animal's body. At least two and preferably an even number of electrodes 241 are used to ensure proper anchoring and also to provide a plurality of interface sites that may be used for electrical stimulation.

With reference to the exemplary implanted stimulator in FIG. 2, a plurality of lead anchor points is chosen so that interface site does not need to be predetermined, but rather programmably chosen or changed at the time of stimulation. The present invention provides a means to dynamically select electrodes for tissue interfacing. A plurality of electrodes 301-308 are anchored in body lumens 258 and 259 and are connected to the insulated conductors 300 to the selection logic 60 that is programmably controlled by the control circuit 230. For example, the controller 56 monitors each electrode termini 301-308 and selects an electrode combination that that can provide optimal stimulation. The controller 56 also senses anatomical electrical signals at the electrode sites and responds by choosing appropriate sites for optimizing stimulation.

In one case, contact electrodes 301 and 302 are optimally chosen through the selection logic 60 for stimulating the tissue. Here the stimulation voltage waveform produces by the stimulation signal generator 58 is routed by the selection logic 60 to those selected contact electrodes 301 and 302. The polarity of these contact electrodes chosen by the selection logic 60 as well. In one instance, electrode 301 is the positive contact electrode and electrode 302 is the negative counterpart. In another instance, the polarity of contact electrodes 301 and 302 is reversed. It should be noted that unipolar, bipolar and multi-polar electrical stimulation can be employed. At other times, other pair combinations of contact electrodes, e.g. contact electrodes 303 and 304 or 302 and 306, are chosen based on their proximity to the desired stimulation site.

In some embodiments contemplated in the present invention, certain contact electrodes can be turned on for stimulating tissue in a programmed sequence. This kind of sequencing can be used to perform muscle or neuronal activation. As an example, contact electrode pairs 301 and 302 are on for a preset time, followed by contact electrode pairs 302 and 303, followed by 303 and 304. This sequence can be repeated for a preset amount of time or preset number of times.

It should be noted that different stimulation protocols can be employed with the multiple electrodes available for selection. Each stimulation protocol includes specifying waveforms for stimulation, duty cycles, durations, amplitudes, shapes of waveforms, and spatial and temporal sequences of waveforms. The protocols are programmably selected by the control circuit and commands are issued to the stimulation circuitry including multiple electrodes in a deployed state in the lumen. The multiple electrode configuration also allows for different types of stimulation to be carried out concurrently or in an alternating fashion.

A greater number of anchor points further improves securing the lead in the lumen. The anchored electrical interface can then be used for several purposes. In one case, as described earlier, it can be used for programmable transvascular stimulation. In another case, it can be used for sensing electrical signals at the site of deployment. For example, a cardiac lead interface may be used as ECG sensing electrodes. A brain lead interface may be used as EEG sensing electrodes. Similarly, other electrical signals may be sensed using the interface. In some cases, concurrent sensing and stimulation can be provided using the same sets of electrodes. In other instances, sensing and stimulation electrodes may be different. In one embodiment, electrodes may be adapted to stimulate a single site with multiple electrodes. In another embodiment, electrodes may be adapted to stimulate multiple sites with multiple electrodes. In a further embodiment, stimulation sequence and/or duration in multiple distributed electrodes may be spatially and/or temporally varied. In yet another embodiment, stimulation site may be dynamically determined adaptively by sensing responses from multiple sites and selecting the most responsive site. This kind of dynamic determination may be repeated after certain amount of time. In some embodiments of the current invention, sensed outputs of all the applicable electrodes may be analyzed before choosing the signals from best electrodes. In some embodiments, electrode sites making the best contact may be chosen for stimulation and/or sensing.

Using the above characteristics, in general, a self-anchoring lead for providing an electrical interface with a lumen of an animal body contains a lead structure to be implanted inside the lumen. This lead structure has at least two insulated conductors, each of which is connected to an electrode that has an associated shape memory material and a rounded terminus to grip the lumen wall for anchoring the lead. A separate conductor connects each electrode to a control circuit wherein the control circuit programmably selects electrodes for electrically interfacing with the lumen.

More specifically, the self-anchoring lead electrically interfaces with a blood vessel in an animal. This lead includes a plurality of insulated conductors that preferably are coiled about a common axis as shown in the FIG. 5, however they may be coiled along different axes. Each insulated conductor is connected to an electrode and has an associated shape memory material. The electrode has a rounded terminus to grip the blood vessel wall for anchoring the lead when released from a sheath that holds the electrode in a collapsed state. The lead structure has an internal lumen for receiving a guidewire or any other placement aid. Finally, the components of the lead may be encased in an external biocompatible layer.

In order to implant the self-anchoring lead in a lumen of the animal's body, the self-anchoring lead is provided in collapsed state in which the electrode termini are confined close to the longitudinal axis of the lead. Preferably a removable sheath is employed to confine the electrode termini in this manner. The distal end of the lead is inserted into the animal through an opening in the lumen and advanced along the lumen until the expandable portion with the electrode termini is adjacent the desired interface site. Then, the expandable portion of the lead is released, or deployed, into the expanded state, such as by sliding a sheath that retained the electrodes in a collapsed state. In the deployed state, rounded termini grip the lumen wall, thereby anchoring the lead.

As mentioned previously above, several variations of the basic electrode configurations can be used for tissue stimulation of various organs in animals. In fact, the device can be scaled appropriately to be applicable to be placed in any lumen for stimulation purposes and not just limited to the vascular system. Therefore, the scope of the electrode configurations should be viewed to encompass all such endoluminal prosthetic alternatives.

The foregoing description was primarily directed to preferred embodiments of the invention. Even though some attention was given to various alternatives within the scope of the invention, it is anticipated that one skilled in the art will likely realize additional alternatives that are now apparent from disclosure of embodiments of the invention. Accordingly, the scope of the invention should be determined from the following claims and not limited by the above disclosure.

1. An apparatus for providing an electrical interface with a lumen of a body of an animal, said apparatus comprising:
a self-anchoring electrical lead for implantation inside the lumen and having at least two insulated conductors, each of which being connected to a separate electrode that has shape memory material and a rounded terminus for engaging a wall of the lumen to anchor the lead; and
a stimulation circuit connected to the at least two insulated conductors and generating a stimulation voltage and selecting a pair of the plurality of electrodes to which the stimulation voltage is applied stimulate tissue of the wall of the lumen.

2. The apparatus as recited in claim 1 wherein a diameter of the rounded terminus of the electrode is greater than a diameter of the respective insulated conductor.

3. The apparatus as recited in claim 1 wherein the lumen is a blood vessel.

4. The apparatus recited in claim 1 wherein the self-anchoring electrical lead further comprises a moveable sheath that in a first position encases each electrode in a contracted state and in a second position releases each electrode into an expanded state.

5. The apparatus as recited in claim 1 wherein the shape memory material is one of Nitinol, stainless steel, a nickel-cobalt based alloy, a shape memory polymer, and a shape memory ceramic adjacent to the associated insulated conductor.

6. The apparatus as recited in claim 1 wherein the shape memory material is is one of a stainless steel conductor and a nickel-cobalt alloy conductor.

7. The apparatus as recited in claim 1 wherein the self-anchoring electrical lead further comprises an internal lumen for receiving a work implement.

8. The apparatus as recited in claim 1 wherein the self-anchoring electrical lead further comprises an outer layer of a biocompatible material.

9. The apparatus as recited in claim 1 wherein the shape of the rounded terminus is one of spherical, capsule-like and ellipsoidal.

10. A self-anchoring lead for providing an electrical interface with a blood vessel of an animal, said self-anchoring lead comprising:
an electrical lead for implantation inside the blood vessel with a plurality of coiled insulated conductors, each of which is connected to a separate electrode that has shape memory material and a rounded terminus for engaging a wall of the blood vessel to anchor the lead, the electrical lead further comprising a sheath that is slideable along the exterior of the plurality of coiled insulated conductors from a first position that encases each electrode in a contracted state to a second position where each electrode is released into an expanded state in which each rounded terminus engages the wall of the blood vessel.

11. The self-anchoring lead as recited in claim 10 wherein the electrical interface provides transvascular stimulation therapy to the wall of the blood vessel.

12. The self-anchoring lead as recited in claim 10 wherein the electrical interface provides transvascular sensing of electrical parameters from the wall of the blood vessel.

13. The self-anchoring lead as recited in claim 10 wherein the shape of the rounded terminus is one of spherical, capsule-like and ellipsoidal.

14. The self-anchoring lead as recited in claim 10 wherein the shape memory material is one of Nitinol, stainless steel, and a nickel-cobalt based alloy adjacent to the associated insulated conductor.

15. The self-anchoring lead as recited in claim 10 wherein the shape memory material is one of a stainless steel conductor and a nickel-cobalt alloy conductor.

16. A method of providing an electrical interface with a lumen in a body of an animal, said method comprising:
providing self-anchoring lead structure which has a expandable portion that has a plurality of electrodes each having a shape memory material and a rounded terminus for engaging a wall of the lumen to anchor the lead, a non-expandable portion comprising a plurality of coiled, insulated conductors connected to each of the electrodes, and a sheath releasably holding the plurality of electrodes in a contracted state;
implanting the self-anchoring lead structure in a collapsed state by inserting the lead through an opening in the lumen and advancing the lead through the lumen to a desired interface site; and
sliding the sheath to release the expandable portion of the lead structure to attain an expanded state in which the plurality of electrodes engage the lumen wall and anchor the lead; and
programmably selecting electrodes for electrically interfacing with the lumen using a control circuit connected to the plurality of electrodes.

17. The method as recited in claim 16 further comprises electrically stimulating tissue in the animal by transluminal stimulation.

18. The method as recited in claim 16 further comprises electrically sensing physiological characteristics in the animal.

19. The method as recited in claim 16 wherein the shape of the rounded terminus is one of spherical, capsule-like and ellipsoidal.

20. The method as recited in claim 16 wherein the shape memory material is a Nitinol wire adjacent to the associated insulated conductor.
